(19) United States
(12) Patent Application Publication
(10) Pub. No.: US 2020/0237856 A1
(43) Pub. Date: Jul. 30, 2020

(54) METHOD OF TREATING PATIENTS WITH HEPATORENAL SYNDROME TYPE 1

(71) Applicant: Mallinckrodt Hospital Products IP Limited, Dublin (IE)

(72) Inventors: Khurram Jamil, Yardley, PA (US); Stephen Chris Pappas, The Woodlands, TX (US); Jim Potenziano, Binghamton, NY (US)

(21) Appl. No.: 16/828,681

(22) Filed: Mar. 24, 2020

Related U.S. Application Data
(63) Continuation-in-part of application No. 16/669,151, filed on Oct. 30, 2019.
Application No. 16/669,151 is a continuation-in-part of application No. 16/411,944, filed on May 14, 2019.
Application No. 16/411,944 is a division of application No. 14/920,392, filed on Oct. 22, 2015, now Pat. No. 10,335,452.
(60) Provisional application No. 62/068,357, filed on Oct. 24, 2014.
(60) Provisional application No. 62/151,384, filed on Apr. 22, 2015.

(51) Int. Cl. A61K 38/095; G01N 33/49; G01N 15/10; A61M 27/00; A61K 45/06; A61K 38/38; A61K 9/00; A61B 5/145; A61B 5/0205

(52) U.S. Cl. CPC A61K 38/095; G01N 33/4925; G01N 15/10; A61M 27/002; A61K 45/06; G01N 2015/008; A61K 9/0019; A61B 5/14542; A61B 5/0205; G01N 2015/1062; A61K 38/38

(57) ABSTRACT

The principles and embodiments of the present disclosure relate to methods for using terlipressin to treat a patient having impaired renal function associated with liver disease. A method of treating an adult patient with type 1 hepatorenal syndrome (HRS-1) may include assessing a baseline serum creatinine level prior to administration of terlipressin to the patient, initiating dosing of about 0.5 mg to about 1 mg of terlipressin to the patient every 6 hours by IV for 1-3 days, assessing a serum creatinine level in the patient at day 4±1 day from initiating dosing, administering a modified dosage of terlipressin based on a comparison of the assessed serum creatinine level at day 4±1 day and the baseline serum creatinine level, and continuing administration until 24 hours after two consecutive serum creatinine levels of ≤1.5 mg/dL at least 2 hours apart for a maximum of 14 days. The treatment may result in verified reversal of the HRS-1.

### CLAIM OF PRIORITY

This application is a continuation-in-part of U.S. patent application Ser. No. 16/669,151, filed on Oct. 30, 2019, which is a continuation-in-part of U.S. patent application Ser. No. 16/411,944, filed on May 14, 2019, which is a divisional application of U.S. patent application Ser. No. 14/920,392, filed on Oct. 22, 2015, which claims priority under 35 USC § 119(e) to U.S. Patent Application Ser. No. 62/151,384, filed on Apr. 22, 2015, and U.S. Patent Application Ser. No. 62/068,357, filed on Oct. 24, 2014, the entire contents of which are hereby incorporated by reference.

### INCORPORATION OF SEQUENCE LISTING

A computer readable text file, entitled “620746_SequenceListing_ST25.txt” created on or about Jul. 10, 2019, with a file size of about 1 kilobyte contains the sequence listing for this application and is hereby incorporated by reference in its entirety.

### TECHNICAL FIELD

Principles and embodiments of the present disclosure relate generally to methods of treating patients with type-1 hepatorenal syndrome.

### BACKGROUND

Hepatorenal Syndrome Type-1 (HRS Type 1 or HRS-1) is the development of acute kidney failure in patients with late-stage liver cirrhosis in the absence of any other cause. It is characterized by rapid onset of renal failure with a high mortality rate that exceeds 80% with within three months. Renal failure is an identified complication of cirrhosis of the liver; and, acute renal failure is known to have poor prognosis for patients with cirrhosis of the liver. In various instances, the renal failure may be caused by hypovolemia, hepatorenal syndrome without ongoing infection, or hepatorenal syndrome with an ongoing infection. Unfortunately, patients with HRS Type-1 may die from renal failure while waiting for a liver transplant. Currently, there is no way of determining which patients could maximally benefit from terlipressin treatment to reverse HRS Type-1.

Hepatorenal Syndrome (HRS) is indicated by low glomerular filtration rate due to renal vasoconstriction, splanchnic and peripheral arterial vasodilatation producing decreased vascular resistance, and portal hypertension. HRS is indicated by cirrhosis with ascites, serum levels of creatinine>133 μmol/l (1.5 mg/dL), no improvement of serum levels of creatinine (decrease to a level of ≤133 μmol/l) after at least 2 days of diuretic withdrawal and volume expansion with albumin, and the absence of shock and parenchymal kidney disease. HRS Type 1 is indicated by doubling of the initial serum levels of creatinine to >226 μmol/l (2.56 mg/dL) in <2 weeks.

Normal creatinine levels range from 0.7 to 1.3 mg/dL in men and 0.6 to 1.1 mg/dL in women. One mg/dl of creatinine equals 88.4 μmol/l.

Certain mechanisms that work to maintain effective arterial blood volume and relatively normal arterial pressure in patients with cirrhosis, however, affect kidney function, such as sodium and solute-free water retention, which can lead to ascites and edema, and to renal failure by causing intrarenal vasoconstriction and hypoperfusion. Acites can result from the combination of portal hypertension and splanchnic arterial vasodilation that alters intestinal capillary pressure and permeability, which facilitates the accumulation of the retained fluid in the abdominal cavity.

A factor contributing to ascites formation is a splanchnic vasodilation that results in a decreased effective arterial blood volume. Portal hypertension also results from increased hepatic resistance to portal blood flow in cirrhotic livers, and may induce splanchnic vasodilation. There may be a marked impairment in solute-free renal water excretion and renal vasoconstriction, which leads to HRS.

In various instances, there may be signs of hepatic decompensation including INR>1.5, ascites, and encephalopathy. Hyponatremia is also a frequent complication of patients with cirrhosis and ascites that is associated with increased morbidity.

Systemic Inflammatory Response Syndrome (SIRS) is an inflammatory response that is not necessarily related to an infection, but may be due to nonspecific insults that initially produces local cytokines. SIRS is typically characterized by four criteria, including (1) core body temperature of less than 36° C. (96.8° F.) or greater than 38° C. (100.4° F.), (2) a heart rate of greater than 90 beats per minute, (3) tachypnea (high respiratory rate), with greater than 20 breaths per minute; or, an arterial partial pressure of carbon dioxide (CO2) of less than 4.3 kPa (32 mmHg), and (4) a white blood cell count less than 4000 cells/mm³ (4×109 cells/L) or greater than 12,000 cells/mm³ (12×109 cells/L); or the presence of greater than 10% immature neutrophils (band forms) band forms greater than 3% is called bandemia or a “left-shift.” SIRS can be diagnosed when two or more of these criteria are present.

Sepsis has been defined as a systemic inflammatory response to infection, and septic shock is sepsis complicated by either hypotension that is refractory to fluid resuscitation or by hyperlactatemia.

The mortality of patients suffering from HRS and SIRS can be quite high, approaching 70%.

A number of studies have been conducted on patients having end-stage liver disease and systemic inflammatory responses. One such study described by Thabut et al., disclosed in HEPATOLOGY, Vol. 46, No. 6, 2007 entitled “Model for End-Stage Liver Disease Score and Systemic Inflammatory Response Are Major Prognostic Factors in Patients with Cirrhosis and Acute Functional Renal Failure”, which is incorporated herein by reference in its entirety, concluded that the presence of SIRS criteria with or without infection was a major independent prognostic factor in patients with cirrhosis and acute functional renal failure.

The presence of HRS and SIRS typically indicates a short life span if not effectively treated with the proper medication within a short span of time. It is therefore paramount that the most effective treatments for patients presenting with particular symptoms be identified and the patients started on an appropriate regimen as quickly as possible.

Terlipressin is a synthetic analogue of vasopressin having a prolonged effect, which acts as a peptidic vasopressin Vla receptor agonist. Terlipressin is a derivative of vasotocin prepared by extending the N-terminal by three amino acid residues, and used as a vasoactive drug in the management of hypotension. Terlipressin may be synthesized by coupling amino acids stepwise to one another in a liquid or solid phase with a peptide synthesizer. Terlipressin is a prodrug that slowly metabolizes to lysine-vasopressin and in this way provides prolonged biological effect. The half-life of terlipressin is 6 hours (the duration of action is 2-10 hr), as opposed to the short half-life of vasopressin, which is only 6 minutes (the duration of action is 30-60 min).

The chemical structure for terlipressin (Gly-Lys-Pro-Cys-Asn-Gln-Phe-Tyr-Cys-Gly-Gly-Gly; SEQ ID NO: 1) in an injectable formulation is show below.

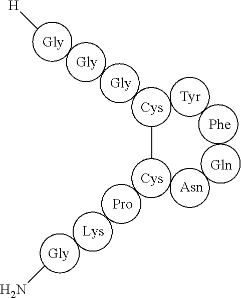

Molecular Formula: C₅₂H₇₄N₁₆O₁₅S₂

Molecular Weight: 1227.4 daltons

Appearance: Homogenous lyophilized white to off-white solid

Solubility: Clear, colorless solution in saline

Vials: Colorless glass vials containing 11 mg of a white to off-white solid, 1 mg active ingredient and 10 mg mannitol.

The active ingredient, N-[N-(N-glycylglycyl)glycyl]-8-L-lysinevasopressin, is a synthetically manufactured hormonogen of 8-lysine vasopressin, composed of 12 amino acids and having the characteristic ring structure of a cyclic nonapeptide with a disulfide bridge between the fourth and the ninth amino acid. Three glycyl-amino acids are substituted at position 1 (cysteine) of 8-lysine-vasopressin. By this N-terminal extension of 8-lysine-vasopressin the metabolic degradation rate of the active ingredient is significantly reduced, because the glycyl molecules inhibit rapid N-terminal enzymatic degradation. Terlipressin may be present in pharmaceutical compositions as a salt, diacetate salt, hydrate, and/or free base, such as terlipressin acetate or terlipressin diacetate pentahydrate.

### SUMMARY

Principles and embodiments of the present disclosure relate generally to methods of treating patients having HRS-1 by administering terlipressin to the patients to obtain reversal of the HRS-1. In one or more embodiments, response criteria provide a new and useful function of indicating a likelihood of improved response by a patient to the administration of terlipressin.

Some aspects of the disclosure relate to a method of reversing type 1 hepatorenal syndrome (HRS-1), where the method includes administering, to a patient having HRS-1, about 0.5 mg to about 1 mg of terlipressin every 6 hours for up to 3 days; measuring serum creatinine in the patient after 3 days administration; and comparing the measured serum creatinine to a baseline serum creatinine level. If serum creatinine decreased by at least 30%, continue administering about 0.5 mg to about 1 mg terlipressin every 6 hours. If serum creatinine has not decreased by 30%, administer about 1 mg to about 2 mg of terlipressin every 6 hours. If serum creatinine is at or above the baseline serum creatinine level, discontinue administering terlipressin. In some examples, the terlipressin administered may be terlipressin acetate. The patient may not have RRT post-liver transplant for at least 10 days to at least 30 days after starting administering the terlipressin.

Additional aspects of the disclosure relate to methods of treating type 1 hepatorenal syndrome (HRS-1). The method may include identifying a patient as having HRS-1; administering, to a patient having HRS-1, about 0.5 mg to about 1 mg of terlipressin every 6 hours for up to 3 days; measuring serum creatinine in the patient after 3 days administration; and comparing the measured serum creatinine to a baseline serum creatinine level. If serum creatinine decreased by at least 30%, continue administering about 0.5 mg to about 1 mg terlipressin every 6 hours. If serum creatinine has not decreased by 30%, administer about 1 mg to about 2 mg of terlipressin every 6 hours. If serum creatinine is at or above the baseline serum creatinine level, discontinue administering terlipressin. In some examples, the terlipressin administered may be terlipressin acetate. The patient may not have RRT post-liver transplant for at least 10 days to at least 30 days after starting administering the terlipressin.

Other aspects of the disclosure relate to methods of reversing type 1 hepatorenal syndrome (HRS-1), including assessing a baseline serum creatinine level prior to administration of terlipressin to the patient; initiating dosing of about 0.5 mg to about 1 mg of terlipressin to the patient every 6 hours by IV for 1-3 days; assessing a serum creatinine level in the patient at day 4±1 day from initiating dosing; and administering a modified dosage of terlipressin based on a comparison of the assessed serum creatinine level at day 4±1 day and the baseline serum creatinine level. The modified dosage may be about 0.5 mg to about 1 mg terlipressin every 6 hours if serum creatinine decreased by at least 30%. The modified dosage may be about 1 mg to about 2 mg terlipressin every 6 hours if serum creatinine has not decreased by 30%. The modified dosage may be a discontinuation of administering terlipressin if serum creatinine is at or above the baseline serum creatinine level. In some examples, the terlipressin administered may be terlipressin acetate. The patient may not have RRT post-liver transplant for at least 10 days to at least 30 days after starting administering the terlipressin.

Additional aspects and features are set forth in part in the description that follows, and will become apparent to those skilled in the art upon examination of the specification or may be learned by the practice of the disclosed subject matter.

### BRIEF DESCRIPTION OF THE DRAWINGS

Further features of embodiment of the present disclosure, their nature and various advantages will become more apparent upon consideration of the following detailed description, taken in conjunction with the accompanying drawings, which are also illustrative of the best mode contemplated by the applicants, and in which like reference characters refer to like parts throughout, where:

FIG. 1 illustrates an exemplary embodiment of a terlipressin treatment protocol;

FIG. 2 illustrates an exemplary embodiment of a terlipressin treatment protocol;

FIG. 3 illustrates a set of unexpected results from an exemplary embodiment of a terlipressin treatment protocol; and

FIG. 4 illustrates an exemplary embodiment of a terlipressin treatment protocol.

### DETAILED DESCRIPTION

The principles and embodiments of the present disclosure relate to methods of improving a patient's renal condition involving a treatment protocol comprising terlipressin. Accordingly, various embodiments of the present disclosure provide methods of treating a patient with terlipressin or terlipressin and albumin.

As used herein, use of “terlipressin” may refer to terlipressin or salts, diacetate salts, hydrates, and/or free bases thereof. For example, use of terlipressin may include terlipressin acetate or terlipressin diacetate pentahydrate. In additional examples, terlipressin may refer to any other suitable salts or hydrates thereof or any other biologically acceptable salts or hydrates thereof.

In embodiments of the present disclosure, the patient is evaluated to determine the particular disease and/or syndrome he or she may be suffering from, and beginning a treatment regimen for patients that will benefit from the administration of terlipressin.

In various embodiments, the patient has end stage liver disease complicated with acute kidney failure, such as HRS, and is treated with terlipressin.

In various embodiments, end-stage liver disease may be cirrhosis of the liver or fulminant liver failure. In various embodiments, the end-stage liver disease is complicated by impaired renal function.

HRS-1 in decompensated cirrhosis is related to hemodynamic abnormalities. Terlipressin improves renal perfusion in HRS-1 by enhancing intravascular volume through splanchnic vasoconstriction. In some aspects, terlipressin may be more effective than placebo in albumin-treated patients with decompensated cirrhosis and HRS-1. An aspect of the present disclosure relates to a method of diagnosis of patients that show improved response to terlipressin treatment, as indicated by an increased probability of HRS reversal.

In one or more embodiments, the method of identifying an HRS-1 patient with an increased likelihood of responding to terlipressin treatment regimen comprises identifying a patient as having end stage live disease and impaired renal function, determining if the patient also exhibits at least two out of three criteria for SIRS, wherein the three response criteria include (1) a white blood cell count (WBC) that is less than 4,000 cells/mm³or greater than 12,000 cells/mm³, (2) a heart rate of greater than 90 beats per minute (BPM), and (3) an HCO3<21 mmol/L, where HCO3 is considered a surrogate measurement that approximates the response criteria of arterial partial pressure of carbon dioxide (PaCO2)<32 mmHg. In various embodiments, a heart rate of >85 BPM and/or an HCO3<23 mmol/L may be applied as the response criteria.

An aspect of the present disclosure relates to terlipressin for use in the treatment of HRS-1 in a subject that is also exhibiting at least two of the following three response criteria:
(a) a white blood cell count (WBC) is less than 4,000 cells/mm³ or greater than 12,000 cells/mm³,
(b) a heart rate of greater than 90 beats per minute (BPM), and
(c) an HCO3<21 mmol/L, where HCO3 is considered a surrogate measurement that approximates the response criteria of arterial partial pressure of carbon dioxide (PaCO2)<32 mmHg. In various embodiments, one or more single dosages of terlipressin is administered to the subject, thereby treating the HRS-1.

In various embodiments, the terlipressin dosage is administered to the patient in the range of about 0.5 mg to about 2.0 mg every 4 to 6 hours, as a series of single doses, so that the patient receives a single dose in the range of about 0.5 mg to about 2.0 mg of terlipressin followed by another single dose 4 to 6 hours later. In various embodiments, a patient may receive 4 to 6 doses over a 24 hour period, where each dose is in the range of about 0.5 mg to about 2.0 mg. In various embodiments, the total dosage does not exceed 4.0 mg over a 24 hour period.

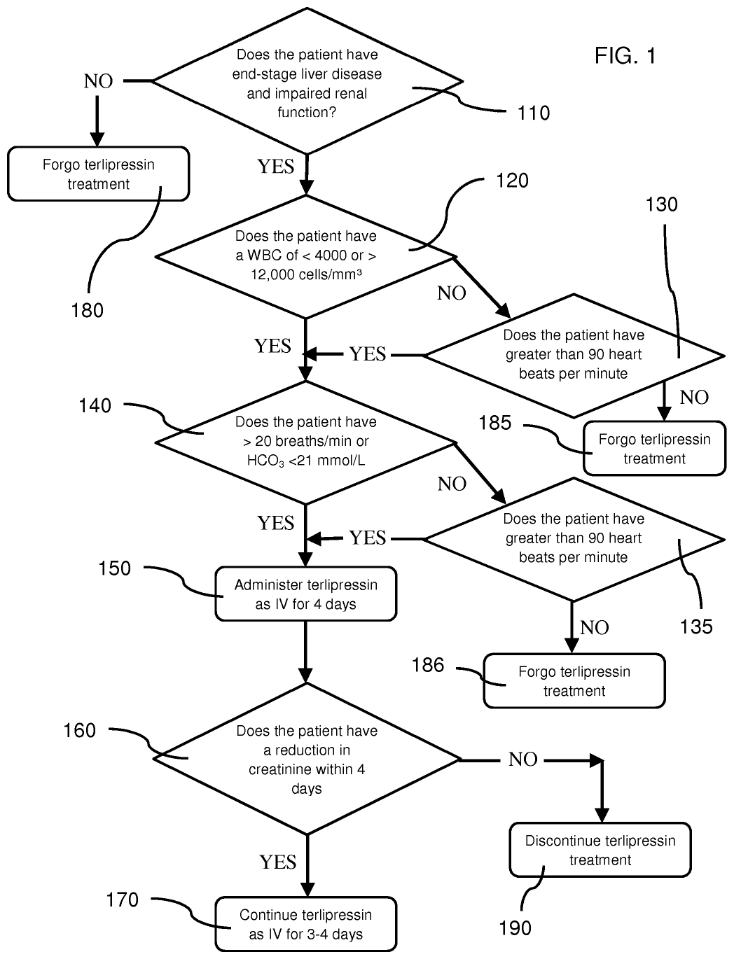

As shown in FIG. 1, an exemplary embodiment of a method of treating a patient via an embodiment of a terlipressin treatment protocol.

In various embodiments, a patient, who is initially identified as having end stage liver disease, for which treatment with a vasodilator may provide an improvement in renal function, is tested to determine the extent of the patient's cirrhosis and renal failure.

At 110, a patient is initially identified as having end stage live disease and impaired renal function. In various embodiments, a patient may be suffering from cirrhosis of the liver or fulminant liver failure, where a patient identified with cirrhosis of the liver may have a Child-Pugh score of A, B, or C. In various embodiments, a patient identified with cirrhosis of the liver that has a Child-Pugh score of B or C may be considered a viable candidate for terlipressin treatment. In various embodiments, a patient identified with cirrhosis of the liver that has a Child-Pugh score of C may be considered a viable candidate for terlipressin treatment. [0062] Various complications of end-stage liver disease, and in particular cirrhosis, are recognized and have a notably poor prognosis.

In one or more embodiments, a treatment protocol comprising dosages of terlipressin surprisingly provides reversal of one or more complicating factors, such as vasodilation, and reduces mortality from the associated complications within a 90 day window starting with treatment.

In one or more embodiments, the terlipressin treatment protocol comprises identifying a patient having end-stage liver disease and impaired renal function, where the identified patient may benefit from a treatment comprising administration of terlipressin, determining if the patient also exhibits at least two out of three response criteria, excluding the patient from administration of terlipressin if the patient exhibits uncontrolled infection, sepsis, or septic shock is excluded from the terlipressin treatment, and initiating terlipressin treatment by administering a daily dosage of terlipressin to the patient in an amount effective to produce an improvement in renal function, wherein an improvement in renal function is indicated by a reduction in SCr of at least 25% from baseline, reversal of HRS (defined as a decrease in SCr level to ≤1.5 mg/dl), and/or confirmed HRS reversal (defined as two serum creatinine values of ≤1.5 mg/dL at least 48 hours apart)).

In one or more embodiments, the patent is alive at day 90 after initiating terlipressin treatment. For example, a patient that experiences HRS reversal, verified HRS reversal, and/or greater than 30% improvement in SCr after receiving terlipressin may have at least a 60%, 65%, or 70% likelihood of being alive at day 90. In other embodiments, the patient is alive and transplant-free at day 90 after initiating terlipressin treatment. For example, a patient that experiences HRS reversal, verified HRS reversal, and/or greater than 30% improvement in SCr after receiving terlipressin may have at least a 35%, 40%, or 45% likelihood of being alive and transplant-free at day 90.

In one or more embodiments, the terlipressin dosage may be in the range of about mg to about 10 mg, or 0.5 mg to about 5.0 mg, or 0.5 mg to about 2.0 mg, or 0.5 mg to about mg, or about 1.0 mg to about 2.0 mg per single administration. In various embodiments, the injections may be administered intravenously as slow bolus injections over 2 minutes, where the dose may be repeated every four to six hours. If on day 4 of therapy (after a minimum of 10 doses), SCr had decreased, but by less than 30% from the baseline value, the dose may be increased to 2 mg every 6 hours (±30 min) (8 mg/day). The dose may not be increased if the subject had coronary artery disease; or in the clinical setting of circulatory overload, pulmonary edema, or treatment-refractory bronchospasm. In various embodiments, if dosing was interrupted due to a non-ischemic adverse event, terlipressin may be restarted at the same or lower dose (i.e., 0.5 to 1 mg q6h).

At 180, a patient that is not diagnosed with an end-stage liver disease and impairment of renal function is excluded from the terlipressin treatment.

In one or more embodiments, the patient is tested for three specific response criteria, where the criteria include a determination of (1) whether the white blood cell count (WBC) is less than 4,000 cells/mm³or greater than 12,000 cells/mm³, (2) whether the patient has a heart rate of greater than 90 beats per minute (BPM), and/or (3) whether the patient has tachypnea with greater than 20 breaths per minute or an HCO₃<21 mmol/L, where HCO3 is considered a surrogate measurement that approximates the response criteria of arterial partial pressure of carbon dioxide (PaCO₂)<32 mmHg. In various embodiments, the response criterion of a patient's core body temperature being less than 36° C. (96.8° F.) or greater than 38° C. (100.4° F.) is not measured or considered in determining if the patient has two or more response criteria. In some examples, the response criteria may be SIRS criteria. In various embodiments, the criteria may be tested in any order.

At 120, a patient is tested to determine if the patient's WBC is <4,000 or >12,000 cells/mm³. In various embodiments, the testing is specifically directed at determining if the patient's leukocytes are less than 4000 cells/mm³ (4×10⁹ cells/L) or greater than 12,000 cells/mm³(12×0⁹ cells/L). In various embodiments, a patient will be considered to meet the response criterion if the patient's WBC is <5,000 or >12,000 cells/mm3. In various embodiments, the patient is not tested for the presence of greater than 10% immature neutrophils (band forms). In various embodiments, the testing method to determine the WBC may be any of the methods known in the art.

If the patient is found to not have a WBC outside the range of 4,000 to 12,000 cells/mm3, the patient may still be diagnosed with SIRS if the patient meets the two other response criteria.

In various embodiments, a patient that has a WBC<4,000 or >12,000 cells/mm³ is considered to meet that response criterion.

At 130, a patient that does not have a WBC outside the range of 4,000 to 12,000 cells/mm3 is tested to determine if the patient's heart rate is >90 BPM. If the patient's heart rate is >90 BPM, the patient will be considered to meet that response criterion. In various embodiments, a patient with a heart rate of >85 BPM will be considered to meet that response criterion. The testing method to determine the patient's heart rate may be any of the methods known in the art.

In various embodiments, a patient that has a WBC outside the range of 5,000 to 12,000 cells/mm³ is tested to determine if the patient's heart rate is >90 BPM. If the patient's heart rate is >90 BPM, the patient will be considered to meet that response criterion. In various embodiments, a patient with a heart rate of >85 BPM will be considered to meet that response criterion.

At 185, a patient that does not exhibit both a WBC<4,000 or >12,000 cells/mm3 and a heart rate that is >90 BPM is considered to not qualify for two of the three response criteria, and therefore does not meet the requirements to be treated with terlipressin. A patient failing to meet at least two of the three response criteria is excluded from the terlipressin treatment. Such a patient may be treated instead with one or more other pharmacological agents such as nor-epinephrine, vasopressin, or a combination of midodrine and octreotide. Alternatively or in addition, any of the following may be used: N-acetylcysteine, misoprostol, and/or BQ123. Another option is transjugular intrahepatic portosystemic shunt (TIPS). Renal support in the form of dialysis is commonly instituted to manage acute fluid overload in HRS-1 patients, particularly if pharmacological therapies fail. The only effective and permanent treatment for end-stage cirrhosis and HRS is liver transplantation.

At 140, a patient that has a WBC outside the range of 4,000 to 12,000 cells/mm³ or a heart rate that is >90 BPM is tested to determine if the patient has >20 breaths per minute or an HCO₃<21 mmol/L. If the patient has >20 breaths per minute or an HCO₃<21 mmol/L, the patient will be considered to meet that response criterion. In various embodiments, a patient with an HCO₃<23 mmol/L will be considered to meet that response criterion. The testing method to determine the patient's breathing rate or HCO₃ may be any of the methods known in the art.

In various embodiments, a patient that has a WBC outside the range of 5,000 to 12,000 cells/mm³ is tested to determine if the patient has a breathing rate that is >20 breaths per minute or an HCO₃<21 mmol/L. If the patient has a breathing rate that is >20 breaths per minute or an HCO₃<21 mmol/L, the patient will be considered to meet that response criterion. In various embodiments, a patient with an HCO₃<23 mmol/L will be considered to meet that response criterion.

In one or more embodiments, if the patient has a WBC outside the range of 4,000 to 12,000 cells/mm³ and the patient has >20 breaths per minute or an HCO₃<21 mmol/L, the patient is considered to qualify for two of the three response criteria, and therefore meets the requirements to be treated with terlipressin unless otherwise excluded.

In one or more embodiments, if the patient has a heart rate that is >90 BPM and the patient has a breathing rate that is >20 breaths per minute or an HCO₃<21 mmol/L, the patient is considered to qualify for two of the three response criteria, and therefore meets the requirements to be treated with terlipressin unless otherwise excluded.

At 135, a patient that has a WBC outside the range of 4,000 to 12,000 cells/mm³, but does not have >20 breaths per minute or an HCO₃<21 mmol/L, is tested to determine if the patient's heart rate is >90 BPM. If the patient's heart rate is >90 BPM, the patient will be considered to meet that response criterion. In various embodiments, a patient with a heart rate of >85 BPM will be considered to meet that response criterion.

In one or more embodiments, in which the patient has a WBC outside the range of 5,000 to 12,000 cells/mm³, but the patient does not have >20 breaths per minute or an HCO₃<21 mmol/L, the patient is tested to determine if the patient's heart rate is >90 BPM. If the patient's heart rate is >90 BPM, the patient will be considered to meet that response criterion. In various embodiments, a patient with a heart rate of >85 BPM will be considered to meet that response criterion.

In one or more embodiments, if the patient has a breathing rate that is >20 breaths per minute or an HCO₃<21 mmol/L a heart rate that is >90 BPM and the patient has a breathing rate that is >20 breaths per minute or an HCO₃<21 mmol/L, the patient is considered to qualify for two of the three response criteria, and therefore meets the requirements to be treated with terlipressin unless otherwise excluded.

At 186, a patient that does not exhibit (1) a breathing rate that is >20 breaths per minute or an HCO₃<21 mmol/L and does not exhibit (2) a heart rate that is >90 BPM is considered to not qualify for at least two of the three response criteria, and therefore does not meet the requirements to be treated with terlipressin. A patient failing to meet at least two of the three response criteria is excluded from the terlipressin treatment. Optional alternative treatments for such a patient are described above.

While the tests for the response criteria have be discussed in a particular order for the exemplary embodiment, the tests may be done in any particular order.

In one or more embodiments, temperature is not a response criterion because patient temperature may not provide an accurate indication of patient response to terlipressin. In various embodiments, patient temperatures are excluded from the set of response criteria.

At 150, a patient that has end stage liver disease with impaired renal function, and qualifies for at least two of the three response criteria, is started on terlipressin. In one or more embodiments, a patient with uncontrolled infection, sepsis, or septic shock is excluded from the terlipressin treatment. In various embodiments, terlipressin is administered to the patient for one to four days. In various embodiments, the patient is administered terlipressin for four days unless the patient experiences an adverse event. In various embodiments, the terlipressin is administered to the patient as an IV drip.

In one or more embodiments, the terlipressin treatment protocol comprises administering a dosage of terlipressin in the range of about 0.1 mg to about 10 mg, or 0.5 mg to about 5.0 mg, or 0.5 mg to about 2.0 mg, or about 0.5 mg to about 1.0 mg, or about 1.0 mg to about 2.0 mg to the patient over about four hours to about six hours as an IV drip.

In one or more embodiments, the patient is administered terlipressin as an IV about every 4 to 6 hours for 1 to 4 days. In various embodiments, the terlipressin may be administered for at least 4 days.

In one or more embodiments, the patient is administered terlipressin as a slow bolus over 2 minutes about every 4 to 6 hours for 1 to 4 days. In various embodiments, the terlipressin may be administered for at least 4 days.

At 160, the patient that is being administered the terlipressin is tested at least once during the one to four day period of administration to determine if the patient is responding to the terlipressin. In various embodiments, the patient may be tested once prior to beginning the administration of the terlipressin to establish a baseline and once during the one to four days of terlipressin administration, or once prior to beginning the administration of the terlipressin to establish a baseline and once at the end of the four days of administration of the terlipressin. In various embodiments, the patient's creatinine levels are measured to determine if there has been a reduction in the patient's serum creatinine, where a reduction in serum creatinine levels of about 1.0 mg/dL or greater, or in the range of about 1.0 mg/dL to about 2.0 mg/dL, or a reduction of about 1.7 mg/dL from the patient's initial baseline value indicates an improvement in renal function and that the patient is responding to the terlipressin.

In various embodiments, improvement in renal function is indicted by a decrease in serum creatinine level of about 25% or about 30% in the patient receiving terlipressin.

In one or more embodiments, a patient may have his or her serum creatinine levels measured once a day or once every other day for each of the four day period after administration of terlipressin has begun, wherein a measurement made on the first day of terlipressin administration may be recorded and used as the baseline creatinine level.

In various embodiments, the method may comprises testing the patient's SCr level during the 1 to 4 days of terlipressin administration and determining if the patient has a reduction in SCr level by the end of the 1 to 4 days of terlipressin administration.

The serum creatinine levels may be measured by any of the methods known in the art, for example, the Jaffe reaction using alkaline picrate.

The GFR may be measured directly by clearance studies of exogenous markers, such as inulin, iohexol, iothalamate, and Cr51-EDTA, or by estimated glomerular filtration rate (eGFR) using creatinine testing methods that are traceable to a reference method based on isotope dilution-mass spectrometry (IDMS).

At 170, a patient that shows a positive response to the administration of the terlipressin evidenced by a reduction in the patient's serum creatinine level is continued on the terlipressin at the dosage in the range of about 0.1 mg to about 10 mg, or 0.5 mg to about 5.0 mg, or 0.5 mg to about 2.0 mg, or about 0.5 mg to about 1.0 mg, or about 1.0 mg to about 2.0 mg. In various embodiments, the dosage administered to the patient may be adjusted based upon the measured serum creatinine level(s). In various embodiments, a patient being administered terlipressin may have their serum creatinine levels monitored for the entire time period that the patient is receiving terlipressin. In one or more embodiments, the patient's serum creatinine level may be tested every day, or every other day, or every third day, or every fourth day, to confirm that the patient is still responding positively to the terlipressin treatment.

In various embodiments, the patient's terlipressin dosage may be increased from about 0.5 mg to about 1.0 mg to about 1.0 mg to about 2.0 mg after 2-3 days of terlipressin administration to the patient if there is <1.5 mg/dL decrease in SCr during the first 2-3 days of treatment.

In various embodiments, the dosage may be repeated every four to six hours for a time period of one or more days until the patient shows recovery, or until the patient no longer shows improvement. The terlipressin may be administered to the patient for a time period in the range of about two days to about sixteen days, or for a time period in the range of about four days to about eight days. In various embodiments, the time period is in the range of about seven days. In various embodiments, the terlipressin treatment may be continued until there is a complete response. In various embodiments, the duration of treatment of a patient with terlipressin may be 1 to 28 days.

At 190, a patient that does not show any improvement by the end of four days may have the terlipressin discontinued, where improvement is indicated by a decrease in serum creatinine levels over the one to four days the terlipressin is administered. In various embodiments the patient may be tested on the third or fourth day after starting treatment with the terlipressin to determine if there is a decrease in serum creatinine levels indicating a response to the treatment.

In one or more embodiments, a patient is provided 2 days of anti-infective therapy for documented or suspected infection before starting administration of terlipressin if an infection is suspected. In various embodiments, a patient may be started on the terlipressin treatment protocol after the patient has been administered the anti-infective therapy.

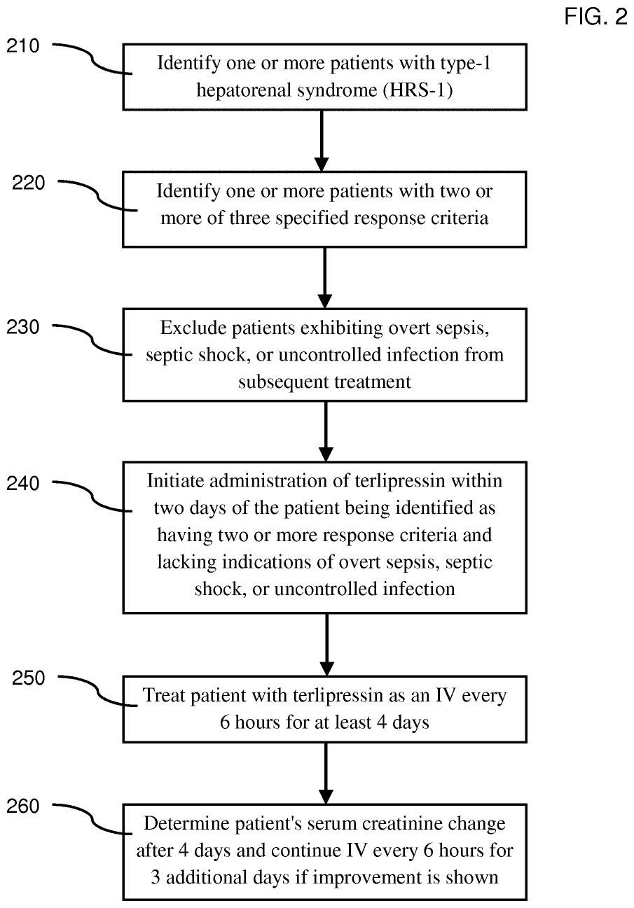

FIG. 2 illustrates an exemplary embodiment of a terlipressin treatment protocol.

Principles and embodiments of the present disclosure also relate to providing terlipressin as an IV every four to six hours to patients that have been identified with HRS-1 and two or more of three specific response criteria.

In one or more embodiments, a patient is tested for (1) a white blood cell count (WBC)<4 or >12 cells/μL; (2) a heart rate (HR)>90 beats per minute (bpm), and (3) HCO3<21 mmol/L.

A non-SIRS patient is defined as subjects with <2 of the response criteria described above.

In various embodiments, temperature is not used as a response criteria.

In one or more embodiments of the disclosure, terlipressin is administered to patients presenting with a particular set of symptoms to mitigate the vasoconstriction in the kidneys, and improve renal function as indicated by a reduction in serum creatinine levels of about 1.7 mg/dL from initial baseline.

At 210, one or more patients that may be presenting with end-stage liver disease are tested to determine whether they are suffering from cirrhosis with ascites, and have serum levels of creatinine>133 μmol/l. A patient identified as having HRS is further tested and/or their medical history checked to determine if the initial serum levels of creatinine have doubled to greater than 226 μmol/l in less than 2 weeks indicating type 1 HRS.

Patients having HRS-1 and at least two of three response criteria have surprisingly shown improved response to terlipressin treatment compared to non-SIRS HRS-1 patients, as indicated by reversal of the HRS indications. In addition, patients having HRS-1, at least two of three response criteria, and not having uncontrolled infection, sepsis, or septic shock have surprisingly shown improved response to terlipressin treatment compared to non-SIRS HRS-1 patients. The HRS indications may include serum creatinine levels.

The patients having HRS-1 and SIRS may experience HRS reversal, verified HRS reversal, or greater than 30% improvement in SCr after receiving terlipressin. In one or more embodiments, the patent is alive at day 90 after initiating terlipressin treatment. For example, a patient that experiences HRS reversal, verified HRS reversal, and/or greater than 30% improvement in SCr after receiving terlipressin may have at least a 60%, 65%, or 70% likelihood of being alive at day 90. In other embodiments, the patient is alive and transplant-free at day 90 after initiating terlipressin treatment. For example, a patient that experiences HRS reversal, verified HRS reversal, and/or greater than 30% improvement in SCr after receiving terlipressin may have at least a 35%, 40%, or 45% likelihood of being alive and transplant-free at day 90.

At 220, once a patient has been identified as suffering from HRS-1, the patient is tested to determine is the same patient is exhibiting at least two out of three criteria indicating SIRS, wherein the three criteria include a (1) WBC<4 or >12 cells/μL; (2) HR>90 bpm, and (3) HCO₃<21 mmol/L.

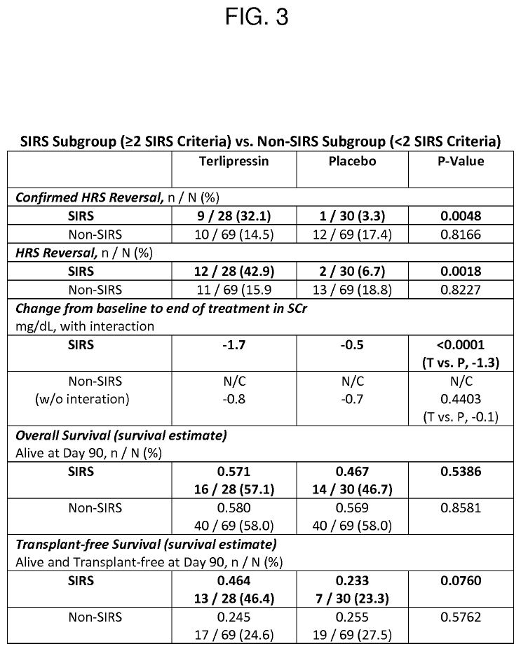

In various embodiments, patients not identified as exhibiting at least two of the three response criteria in addition HRS-1 are excluded from the terlipressin treatment protocol. Patients having HRS-1 and exhibiting at least two of the three response criteria have surprisingly shown improved response to terlipressin treatment compared to non-SIRS HRS-1 patients, as indicated by reversal of the HRS indications, as shown in FIG. 3.

At 230, patients that have been identified as having HRS-1 and exhibit at least two response criteria are tested to determine if they may also have an uncontrolled infection, sepsis, or septic shock, wherein patients identified as exhibiting an uncontrolled infection, sepsis, or septic shock are excluded from the terlipressin treatment protocol.

At 240, patients that have HRS-1, have at least two of the three response criteria, and do not have an uncontrolled infection, sepsis, or septic shock are started on the terlipressin treatment. In one or more embodiments, the terlipressin treatment is started within 48 hours of the initial diagnosis that the patient has both HRS-1 and at least two of three response criteria. In various embodiments, in which the determination that the patient does or does not also have an uncontrolled infection, sepsis, or septic shock occurs after 48 hours of the initial diagnosis of both HRS-1 and the response criteria, the treatment protocol is started within 48 hours of the initial diagnosis, and treatment may be terminated once an uncontrolled infection, sepsis, or septic shock manifests or is determined.

In various embodiments, a baseline serum creatinine level may be determined for the patient prior to starting the administration of terlipressin to the patient; and the administration of terlipressin started within 2 days or within 3 days, or within 4 days of determining the baseline serum creatinine level. In various embodiments, the patient may be tested at least once daily within four days after starting the administration of terlipressin to determine if the patient exhibits a decrease in the serum creatinine level compared to the previously determined baseline serum creatinine level.

At 250, terlipressin treatment of the patient is started and the patient receives a dosage of terlipressin. In one or more embodiments, the terlipressin may be administered to a patient as a slow infusion over 24 hours, wherein the dosage over the 24 hour period may be in the range of about 2.0 mg to about 12 mg. In various embodiments, the dosage over the 24 hour period may be in the range of about 2.0 mg to about 4.0 mg. In various embodiments, the terlipressin is administered as a continuous intravenous (IV) drip lasting from about 4 hours to about 6 hours, and comprising a dosage of about 0.5 mg to about 2.0 mg.

In one or more embodiments, the terlipressin dosage may be a dosage of about 0.5 mg to about 2.0 mg administered intravenously every 4 to 6 hours as a slow bolus injection over 2 minutes.

In one or more embodiments, the terlipressin is used to treat the patient exhibiting HRS-1 and at least two of the three response criteria. In various embodiments, the patient is also tested to determine that the patient does not have an uncontrolled infection, sepsis, or septic shock before being using the terlipressin to treat the HRS-1 patient.

In various embodiments, the terlipressin dosage is given as a continuous IV feed.

In one or more embodiments, the terlipressin dosage is 1 mg administered intravenously every 6 hours as a slow bolus injection over 2 minutes.

In various embodiments, the terlipressin dosage is not given as a bolus.

The terlipressin may be administered to the patient for up to 4 days, wherein the patient may be tested each day of the four days to determine whether the patient is responding to the terlipressin treatment. In various embodiments, a response to the terlipressin treatment may be indicated by a change in the patient's serum creatinine levels, where indication may be a reduction in SCr of at least 25% from baseline. In various embodiments, the terlipressin may be administered for at least 4 days.

At 260, the amount of serum creatinine change is determined after 4 days of treatment with terlipressin, and the treatment with terlipressin continued if the serum creatinine level has improved. In various embodiments, a sufficient improvement in serum creatinine levels after 4 days of treatment is indicated by a decrease of at least 1.0 mg/dL in serum creatinine level, or a decrease of about 1.7 mg/dL in serum creatinine level.

In various embodiments, the patient receives terlipressin for an additional 3 days to 8 days if improvement was exhibited over the previous 1 to 4 days. In various embodiments, the patient receives terlipressin for an additional 3 days to 4 days if improvement was exhibited over the previous 1 to 4 days.

In various embodiments, the administration of terlipressin to the patient is continued for an additional 3 days to 12 days beyond the initial 4 days if the patient exhibits a decrease in the serum creatinine level. In various embodiments, administration of terlipressin to the patient may be continued until at least one SCr value<1.5 mg/dL is obtained. In various embodiments, the duration of treatment may be extend to a maximum of 15 days or 16 days if HRS reversal was first achieved on days 13 or 14, respectively. In various embodiments, the duration of treatment of a patient with terlipressin may be 1 to 28 days. In various embodiments, a decrease in the serum creatinine level may be indicated by a reduction in SCr of at least 1% or of at least 5% or at least 10% or at least 15% or at least 20% or at least 25% from baseline.

In one or more embodiments, the patient may have been administered albumin prior to beginning the terlipressin treatment protocol, and/or prior to the determination that the patient has HRS-1, at least two of the three response criteria. In various embodiments, albumin may be administered to a patient 7 days to 2 days before starting administration of terlipressin to the patient. In various embodiments, the albumin treatment comprises administering 1 gram albumin per 1 kg of patient weight up to a maximum of 100 g per day of albumin to a patient. In various embodiments, albumin may be administered in the range of about 20 g/day to about 50 g/day, where the albumin may be administered for the time period that the patient is administered terlipressin.

A non-limiting embodiment of a method of treating HRS-1 patients exhibiting at least two of three response criteria with terlipressin comprises administering to a patient in need of such treatment a dosage of terlipressin in the range of 2.0 mg to 12.0 mg per day for 1 to 28 days, or in the range of 2.0 mg to 4.0 mg per day for 1 to 7 days, wherein the dosage may be administered as a continuous IV feed or as a slow bolus injection.

Embodiments of the present disclosure also relate to treating patients with HRS-1 and meeting two or more response criteria with one dose of terlipressin every six hours, where the dose is in the range of about 0.5 mg to 2.0 mg, for 3 to 8 days to achieve reversal of the HRS-1.

Embodiments of the present disclosure also relate to initiating terlipressin treatment within 48 hours of determining that a patient is presenting with HRS-1 and at least two of three response criteria, but without sepsis, septic shock, or uncontrolled infection.

Another aspect of the present disclosure relates to a method of distributing a pharmaceutical product.

In one or more embodiments, the method of distributing comprises supplying terlipressin to a medical provider, where the medical provider may be responsible for treating a patient suffering from type 1 hepatorenal syndrome. In various embodiments, the patient does not have overt sepsis, septic shock, or uncontrolled infection. In various embodiments, the method includes providing a recommendation to the medical provider to treat the patient suffering from type 1 hepatorenal syndrome that does not have overt sepsis, septic shock, or uncontrolled infection and having at least two of (1) a white blood cell count (WBC) is less than 4,000 cells/mm³ or greater than 12,000 cells/mm³, (2) a heart rate of greater than 90 beats per minute (BPM), or (3) an HCO3<21 mmol/L, with terlipressin in an amount effective to reduce SCr. In one or more embodiments, the medical provider follow the recommendation and administers a treatment to the patient suffering from HRS-1, but not overt sepsis, septic shock, or uncontrolled infection and having at least two of (1) a white blood cell count (WBC) is less than 4,000 cells/mm³or greater than 12,000 cells/mm³, (2) a heart rate of greater than 90 beats per minute (BPM), or (3) an HCO3<21 mmol/L, with terlipressin in an amount effective to reduce SCr.

The efficacy of terlipressin versus placebo in achieving verified HRS-1 reversal may be more pronounced among the subgroup of patients with systemic inflammatory response syndrome (SIRS). Inflammatory cytokines have been implicated in the pathogenesis of HRS-1. Without being limited to any one theory, terlipressin, through its ability to reduce portal pressure, may decrease the extent of bacterial translocation across the gut wall of patients with decompensated cirrhosis, with consequent reduction in endotoxemia and decrease in the production of pro-inflammatory cytokines, hence making it easier for the patients to respond to the hemodynamic effects of terlipressin.

FIG. 3 shows the unexpected results produced by an exemplary treatment protocol.

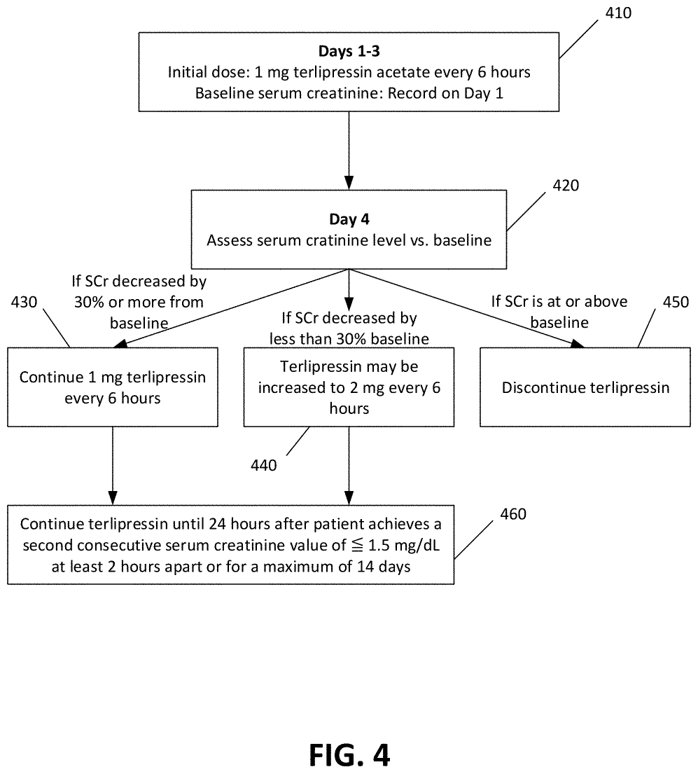

An aspect of the present disclosure relates to methods of treating and/or reversing HRS-1. As shown in FIG. 4, an exemplary embodiment of a method of treating an adult patient with HRS-1 via an embodiment of a terlipressin treatment protocol.

In various embodiments, a patient, who is initially identified as having end stage liver disease, for which treatment with a vasodilator may provide an improvement in renal function, may be tested to determine the extent of the patient's cirrhosis and renal failure. In an embodiment, the patient to be treated is an adult patient that has been diagnosed with HRS-1.

In one or more embodiments, the method of treating an adult patient with type 1 hepatorenal syndrome (HRS-1) includes assessing a baseline serum creatinine (SCr) level prior to administration of terlipressin to the patient, initiating dosing of about 0.5 mg to about 1 mg of terlipressin to the patient every 6 hours by IV for 1-3 days, assessing a serum creatinine level in the patient at day 4±1 day from initiating dosing; and administering a modified dosage of terlipressin based on a comparison of the assessed serum creatinine level at day 4±1 day and the baseline serum creatinine level. In some embodiments, the method may further include continuing administration until 24 hours after two consecutive serum creatinine levels of 1.5 mg/dL at least 2 hours apart for a maximum of 14 days.

In one or more embodiments, the terlipressin dosage may be in the range of about 0.1 mg to about 10 mg, about 0.5 mg to about 10 mg, or 0.5 mg to about 5.0 mg, or 0.5 mg to about 2.0 mg, or 0.5 mg to about 1.0 mg, about 0.85 mg to about 1.7 mg, or about 1.0 mg to about 2.0 mg per single administration.

In an embodiment, the terlipressin administered may be terlipressin acetate. The terlipressin acetate dosage may be administered to the patient in the range of about 0.5 mg to about 2.0 mg. In various examples, the terlipressin acetate dosage may be about 0.5 mg, about 1 mg, about 1.5 mg, about 2 mg, about 2.5 mg, about 3 mg, about 3.5 mg, or about 4 mg.

In various embodiments, the injections may be administered intravenously as slow bolus injections over 2 minutes, where the dose may be repeated every four to six hours. In one or more embodiments, the injections may be administered to the patient over about four hours to about six hours as an IV drip.

In an example, an initial terlipressin dosage is administered to the patient in the range of about 0.5 mg to about 1.0 mg, every 4 to 6 hours, as a series of single doses, so that the patient receives a single dose in the range of about 0.5 mg to about 1.0 mg of terlipressin followed by another single dose 4 to 6 hours later. In various embodiments, a patient may receive 4 to 6 doses over a 24 hour period, where each dose is in the range of about 0.5 mg to about 1.0 mg. In various embodiments, the total dosage does not exceed 4.0 mg over a 24 hour period. In some examples, the terlipressin dosage may be about 0.85 mg or about 1.7 mg terlipressin acetate.

At step 410, in some embodiments, a baseline serum creatinine level may be measured before administration of terlipressin on day 1. Then, an initial dose of terlipressin may be administered to the patient with HRS-1. In an example, the initial dose of terlipressin may about 0.5 mg to about 1.0 mg, and it may be administered every 6 hours for about 1-3 days. In at least one example, the initial dosage may be about 1.0 mg terlipressin acetate (i.e. 0.85 mg terlipressin).

At step 420, on day 4±1 day of administration (e.g. after a minimum of 10 doses), the serum creatinine level may be assessed and compared to the baseline level. In various embodiments, the patient that is being administered the terlipressin is assessed at least once during the days 1 to 4±1 day of administration to determine if the patient is responding to the terlipressin. In various embodiments, the patient may be tested once at the end of 3 or 4 days of administration of the terlipressin. In some examples, the serum creatinine level may be continually assessed (e.g. daily) until administration is discontinued. In various embodiments, the dosage administered to the patient may be adjusted based upon the measured serum creatinine level(s). In various embodiments, a patient being administered terlipressin may have their serum creatinine levels monitored for the entire time period that the patient is receiving terlipressin. In one or more embodiments, the patient's serum creatinine level may be tested every day, or every other day, or every third day, or every fourth day, to confirm that the patient is still responding positively to the terlipressin treatment.

The serum creatinine levels may be measured by any of the methods known in the art, for example, the Jaffe reaction using alkaline picrate. The GFR may be measured directly by clearance studies of exogenous markers, such as inulin, iohexol, iothalamate, and Cr51-EDTA, or by estimated glomerular filtration rate (eGFR) using creatinine testing methods that are traceable to a reference method based on isotope dilution-mass spectrometry (IDMS).

In various embodiments, the patient's creatinine levels are assessed to determine if there has been a reduction in the patient's serum creatinine, where a reduction in serum creatinine levels of about 1.0 mg/dL or greater, or in the range of about 1.0 mg/dL to about 2.0 mg/dL, or a reduction of about 1.7 mg/dL from the patient's initial baseline value indicates an improvement in renal function and that the patient is responding to the terlipressin. In some examples, the assessed serum creatinine level may be 30% or more less than the baseline serum creatinine level, may be between 1% and 29% less than the baseline serum creatinine level, or may be 0% or greater than the baseline serum creatinine level. At steps 430, 440, and 450, a modified dosage of terlipressin may then be administered based on the comparison of the assessed serum creatinine level at day 4±1 day and the baseline serum creatinine level.

At step 430, if the assessed SCr level decreased by 30% or more from the baseline SCr level at day 4±1 day, the about 0.5 mg to about 1.0 mg dosage of terlipressin may be continued to be administered to the patient every 6 hours. For example, the modified dosage may be the same as the initial dosage (e.g. 0.5 mg to 1.0 mg) if the assessed SCr level decreased by 30% or more from the baseline SCr level.

At step 440, if the assessed SCr level has decreased, but by less than 30% from the baseline level at day 4±1 day, the dosage of terlipressin may be increased to about 1.0 mg to about 2.0 mg every 6 hours. For example, the modified dosage may be about 0.1 mg to about 2.0 mg of terlipressin every 6 hours (±30 min) (8 mg/day) if the assessed SCr level has decreased, but by less than 30% from the baseline level. In at least one example, the modified dosage may be The assessed dose may not be increased from the initial dose if the subject had coronary artery disease; or in the clinical setting of circulatory overload, pulmonary edema, or treatment-refractory bronchospasm. In various embodiments, if dosing was interrupted due to a non-ischemic adverse event, terlipressin may be restarted at the same or lower dose (i.e., 0.5 to 1 mg q6h).

At step 450, if the assessed SCr level is at or above the baseline SCr level at day 4±1 day, the administration of terlipressin may be discontinued. For example, the modified dosage may be a discontinuation of administering terlipressin if the assessed SCr level is at or above the baseline SCr level.

At step 460, administration of terlipressin may be continued until 24 hours after the patient achieves a second consecutive serum creatinine value of ≤1.5 mg/dL at least 2 hours apart or for a maximum of 14 days. In various embodiments, the dosage may be repeated every four to six hours for a time period of one or more days until the patient shows recovery, or until the patient no longer shows improvement. In various embodiments, the duration of treatment of a patient with terlipressin may be 1 to 14 days. In various embodiments, the terlipressin may be administered for at least 4 days. In various embodiments, the patient is administered terlipressin for up to 14 days unless the patient experiences an adverse event. In various embodiments, the terlipressin may be administered for at least 3 days, at least 4 days, at least 5 days, at least 6 days, at least 7 days, at least 8 days, at least 9 days, at least 10 days, at least 11 days, at least 12 days, at least 13 days, or at least 14 days. In some examples, the terlipressin may be administered to the patient for a time period in the range of about 2 days to about 14 days, or for a time period in the range of about 4 days to about 8 days. In various embodiments, the time period is in the range of about 7 days. In various embodiments, the terlipressin treatment may be continued until there is a complete response.

In one or more embodiments, a treatment protocol comprising dosages of terlipressin surprisingly provides reversal of one or more complicating factors, such as vasodilation, and reduces mortality from the associated complications within a 90 day window starting with treatment.

Treatment of the patient may include an improvement in renal function. An improvement in renal function is indicated by a reduction in SCr of at least 25% or 30% from baseline, reversal of HRS (defined as a decrease in SCr level to ≤1.5 mg/dl), and/or confirmed HRS reversal (defined as two serum creatinine values of ≤1.5 mg/dL at least 48 hours apart)).

In one or more embodiments, the patent is alive at day 90 after initiating terlipressin treatment. For example, a patient that experiences HRS reversal, verified HRS reversal, and/or greater than 30% improvement in SCr after receiving terlipressin may have at least a 60%, 65%, or 70% likelihood of being alive at day 90. In other embodiments, the patient is alive at day 90 post-liver transplant after initiating terlipressin treatment. For example, a patient that experiences HRS reversal, verified HRS reversal, and/or greater than 30% improvement in SCr after receiving terlipressin may have at least a 35%, 40%, or 45% likelihood of being alive at day 90.

In various embodiments, the adult patient with HRS-1 also is SIRS positive. In one or more embodiments, a patient with uncontrolled infection, sepsis, or septic shock is excluded from the terlipressin treatment.

In one or more embodiments, the patient is also up to a maximum of 100 g per day of albumin each day that the patient is treated with terlipressin. In some examples, the patient may continue to be administered albumin after terlipressin has been discontinued.

The percentage of patients who may achieve verified HRS reversal may be significantly higher with terlipressin than with placebo. In some examples, the patients administered terlipressin may achieve two consecutive SCr values of 1.5 mg/dL or less at least 2 hours apart while receiving treatment by day 14 or discharge. This demonstrates a robust and clinically significant improvement in renal function. In additional examples, the patients administered terlipressin may achieve an absence of renal replacement therapy (RRT) for at least 10 days, which emphasizes the durability of this improvement in renal function. The durability of HRS reversal with terlipressin may also persist to at least day 30 without the need for RRT. In other examples, the patient administered terlipressin may achieve survival for at least 10 days, which establishes the effect of treatment on a key clinical outcome of initial survival. Terlipressin may be superior to placebo in inducing a response across all levels of baseline SCr, with the response rate to terlipressin inversely related to the baseline SCr.

Renal replacement therapy poses particular challenges to patients with HRS-1 and advanced acute-on-chronic liver failure, and the lower rate of RRT and longer survival without RRT in the terlipressin group is clinically relevant. This significantly reduced need for RRT extending into the post-transplant period in the terlipressin group has important clinical implications, as post-transplant RRT is a significant predictor of post-transplant morbidity with worse graft survival mortality, and resource utilization.

### EXAMPLES

### Example 1

A randomized, placebo-controlled, double-blind study was conducted to evaluate the efficacy of terlipressin in HRS type 1. The objective of the study was to determine the efficacy and safety of intravenous terlipressin compared with placebo in the treatment of adult patients with HRS type 1 receiving intravenous albumin. Men and women aged 18 years or older having cirrhosis, ascites, and a diagnosis of HRS type 1 based on the 2007 International Club of Ascites (ICA) diagnostic criteria (Salerno F, Gerbes A, Gines P, Wong F, Arroyo V., Diagnosis, prevention and treatment of hepatorenal syndrome in cirrhosis, Gut. 2007; 56:1310-1318) were eligible for participation. Patients with an SCr level>2.5 mg/dL and either a doubling of SCr within 2 weeks or a change in SCr levels over time indicating a trajectory with a slope equal to or greater than that of a doubling within 2 weeks were enrolled.

Exclusion criteria were intended to product a patient sample limited to individuals with functional renal impairment secondary to cirrhosis and ascites, who could safely be administered terlipressin and who could be expected to survive through the active study period. Among the original exclusion criteria was an exclusion criterion for patients with systemic inflammatory response syndrome (SIRS), defined as the presence of 2 or more of the following findings: (1) temperature>38° C. or <36° C.; (2) heart rate>90/min; (3) respiratory rate of >20/min or a PaCO2 of <32 mm Hg; (4) white blood cell count of >12,000 cells/μL or <4,000/g L. This was based on the concern of enrolling patients with uncontrolled infection. However, it was also recognized that patients with decompensated liver disease frequently have SIRS criteria in the absence of uncontrolled infection or sepsis, and that the presence of 2 or more SIRS criteria is associated with a poor prognosis (Thabut, et al., “Model for End-Stage Liver Disease Score and Systemic Inflammatory Response Are Major Prognostic Factors in Patients with Cirrhosis and Acute Functional Renal Failure,” HEPATOLOGY, Vol. 46, No. 6, December 2007, pp. 1872-1882). Furthermore, the IAC criteria for the definition of HRS type 1 allows for patients with ongoing bacterial infection, but not sepsis or uncontrolled infection, to be considered as having HRS type 1 (as opposed to renal dysfunction associated with infection) (Salerno F, Gerbes A, Gines P, Wong F, Arroyo V., Diagnosis, prevention and treatment of hepatorenal syndrome in cirrhosis, Gut. 2007; 56:1310-1318). The trial protocol required 2 days of anti-infective therapy for documented or suspected infection, allowing enrollment where any SIRS criteria were felt to be most likely explained by underlying hepatic decompensation or other non-infection clinical circumstances. Patients with overt sepsis, septic shock, or uncontrolled infection were excluded. This approach was felt to minimize the chances of enrolling patients at high risk for serious infection while not unduly restricting the enrollment of subjects with HRS type 1.

The patients selected for treatment clinically met the criteria for HRS type 1, where ICA criteria for HRS type 1 allows for patients with ongoing bacterial infection, but not sepsis, to be considered as having HRS type 1, as opposed to renal dysfunction associated with infection. A diagnosis of HRS was not made where the patient remained with obvious manifestations of uncontrolled infection despite antibiotic treatment.

During the active study period treatment with the blinded study drug continued until at least two SCr values<1.5 mg/dL were obtained at least 48 hours apart, or up to 14 days. Duration of treatment was allowed to extend to a maximum of 15 or 16 days if HRS reversal was first achieved on days 13 or 14, respectively. Patients in the active treatment group received terlipressin 1 mg intravenously every 6 hours as a slow bolus injection over 2 minutes. Criteria for dose increases, study discontinuation, treatment resumption and treatment completion during the active study period were provided for. The dosing regimen for patients in the placebo (6 mL lyophilized mannitol solution) group was identical to the terlipressin regimen. The follow-up period began after the end of study treatment and concluded 90 days after the start of study treatment. Survival, renal replacement therapy, and transplantation were assessed.

The SIRS subgroup of patients in this study was defined as any subject with ≥2 of 3 criteria available from the study database which included: (1) WBC<4 or >12 cells/μL; (2) HR>90 bpm and (3) HCO3<21 mmol/L. The latter criterion represented an approximation of the SIRS criterion PaCO2 of <32 mm Hg. This approximation was derived from the observed HCO3 in subjects with HRS in whom a PaCO2 value was available and the calculated HCO3 in subjects with decompensated liver disease and PaCO2 of <32 mm Hg. The non-SIRS subgroup was defined as subjects with <2 criteria described above. Terlipressin response was analyzed in the SIRS and non-SIRS subgroups to determine whether SIRS status had any effect on terlipressin efficacy.

A total of 196 patients were enrolled in the study. Of the 196 patients enrolled, 58 were initially identified as having ≥2 SIRS criteria, including WBC<4 or >12 cells/μL, HR>90 bpm, and HCO3<21 mmol/L, wherein this population was identified as the SIRS subgroup. Based on the criteria defining the SIRS subgroup, baseline WBC and heart rate were slightly higher, and bicarbonate slightly lower, in the SIRS subgroup compared to the non-SIRS and overall study populations. The results of the analysis are shown in FIG. 3.

It was also recognized that patients with decompensated liver disease frequently have SIRS criteria in the absence of uncontrolled infection or sepsis, and that the presence of two or more SIRS criteria is associated with a poor prognosis.

In one or more embodiments, reversal of HRS is indicated by a decrease in SCr level to ≤1.5 mg/dl, and confirmed reversal of HRS is defined as two SCr values of ≤1.5 mg/dL at least 48 hours apart.

As shown in FIG. 3, patients identified as having HRS-1 and at least two of the three criteria for SIRS on a terlipressin treatment protocol exhibited a statistically significant increase in confirmed reversal of HRS (32.1% vs. 3.3%, p<0.005), HRS reversal (42.9% vs. 6.7%, p<0.002) and renal function (change from baseline in SCr, mg/dL, −1.7 vs. −0.5, p<0.0001) compared to placebo. In contrast, in the group of patients having HRS-1 and fewer than two of the SIRS criteria, confirmed reversal of HRS vs. placebo was 14.5% vs. 17.4%, HRS reversal vs. placebo was 15.9% vs. 18.8%, and renal function change vs. placebo was −0.8 vs. −0.7 mg/dL. These results indicate that the presence of two or more of the SIRS criteria indicates that the patient is more likely to have a positive response to treatment with terlipressin.

In addition, in the treatment groups, patients with HRS-1 and two or more SIRS criteria showed an overall survival rate comparable to patients that were suffering from HRS-1, but did not have at least two of the three criteria for SIRS (57.1% vs. 58%).

### Example 2

A randomized, placebo-controlled, double-blind study was conducted to evaluate the efficacy of terlipressin in HRS type 1. The objective of the study was to characterize the efficacy and safety of terlipressin plus albumin versus albumin alone for the treatment of HRS-1 in patients with well-defined HRS-1. The study used the similar inclusion and exclusion criteria as described in Example 1.

In particular, HRS-1 was defined based on modified prior criteria outlined by the International Club of Ascites (ICA), as rapidly deteriorating renal function to SCr≥2.25 mg/dL, with actual or projected doubling of SCr within 2 weeks, without improvement in renal function (<20% decrease in SCr 48 hours after both diuretic withdrawal and albumin-fluid challenge) in adult patients with cirrhosis and ascites. Subjects were randomized 2:1 to terlipressin (1 mg IV every 6 hours) or placebo, plus albumin in both groups. Treatment was continued to Day 14 unless the following occurred: verified HRS reversal (VHRSR), renal replacement therapy (RRT), liver transplantation (LT) or SCr at or above baseline (BL) at Day 4. VHRSR, the primary endpoint, was defined as 2 consecutive SCr values≤1.5 mg/dL, at least 2 hours apart, with subjects alive without RRT for at least 10 days after the second SCr≤1.5 mg/dL; HRS reversal (HRSR) was a decrease in SCr to ≤1.5 mg/dL. Secondary end points included HRS reversal (any SCr value 1.5 mg/dL or less during treatment), HRS reversal without RRT by day 30, HRS reversal in patients with systemic inflammatory response syndrome, and verified HRS reversal without recurrence by day 30.

The patients were at least 18 years of age, with cirrhosis, ascites, and rapidly progressive renal failure, with a SCr doubling to at least 2.25 mg/dL within 14 days. Major exclusion criteria included SCr of greater than 7.0 mg/dL, one or more large-volume paracenteses of 4 L or more within 2 days of randomization, evidence of parenchymal renal diseases or obstructive uropathy, or presence of sepsis and/or uncontrolled bacterial infection. Patients with severe cardiovascular disease or recent (within 4 weeks) renal replacement therapy (RRT) were excluded.

300 subjects were enrolled in the study. Of the 300 subjects, 199 were randomized to terlipressin and 101 to placebo (albumin alone). Patients were stratified by qualifying SCr (less than 3.4 mg/dL or 3.4 mg/dL or greater) and pre-enrollment large-volume paracentesis (at least one single event of 4 L or greater, or less than 4 L within 3 to 14 days prior to randomization). Concomitant albumin was administered in 82.9% of patients in the terlipressin group (165 of 199; mean [SD] total dose of 199.4 [146.8] g) versus 91.1% (92 of 101; mean [SD] dose of 239.5 [183.6] g) in the placebo group (P=0.06). One hundred forty-five patients (72.9%) in the terlipressin group and 72 (71.3%) in the placebo group had received prior midodrine and octreotide.

Demographic and BL clinical characteristics were similar between treatment groups. For example, the two treatment groups had similar average age, weight, height, sex distribution, ethnicity distribution, race distribution, presence of alcoholic hepatitis, baseline serum creatinine, large volume paracentesis (LVP) randomization strata, baseline model end stage liver disease (MELD) score, baseline Child-Pugh score, baseline white blood cell count, baseline bilirubin, baseline mean arterial pressure (MAP), baseline heart rate, baseline blood urea nitrogen (BUN), baseline bicarbonate (HCO₃) or carbon Dioxide (CO₂), baseline temperature, baseline respiratory rate, baseline acute on chronic liver failure (ACLF) grade, baseline chronic liver failure-sepsis organ failure assessment (CLIF-SOFA) score and presence of prior conditions/treatments such as esophageal variceal hemorrhage (EVH) banding, pneumonia, urinary tract infection (UTI), spontaneous bacterial peritonitis (SBP), and receipt of albumin. The proportion of patients in each group who underwent LT was 23.1% for terlipressin and 28.7% for placebo.

A baseline SCr value was assessed before the patients received the assigned treatment. Patients received blinded assigned treatment (terlipressin or placebo) 1 mg administered intravenously over 2 minutes every 6 hours (±30 minutes). In keeping with current guidelines, it was strongly recommended that albumin (1 g/kg to a maximum of 100 g, on day 1 and 20 to 40 g/day thereafter) be administered to all subjects. If SCr decreased <30% from the baseline value on Day 4, after a minimum of 10 doses of study drug, dose increase to 2 mg every 6 hours (±30 minutes) (8 mg/day) was mandated, except in subjects with coronary artery disease or in the setting of circulatory overload, pulmonary edema, or bronchospasm. Dose resumption was permitted after interruption for adverse events except for cardiac or mesenteric ischemia, for which treatment was permanently discontinued.

The primary efficacy end point was the incidence of verified HRS reversal, defined as the percentage of patients with two consecutive SCr values no greater than 1.5 mg/dL at least 2 hours apart, while remaining alive without RRT for at least 10 days after achieving verified HRS reversal, while excluding SCr values after RRT, transjugular intrahepatic portosystemic shunt, liver transplant, or open-label vasopressor from primary end point analysis. 58 patients (29.1%) treated with terlipressin achieved verified HRS reversal versus 16 (15.8%) treated with placebo (P=0.01).

Secondary efficacy end points included incidence of HRS reversal, defined as the percentage of patients with an on-treatment SCr value of 1.5 mg/dL or less; durability of HRS reversal, defined as the percentage of patients with HRS reversal without RRT to day 30; incidence of HRS reversal among patients with systemic inflammatory response syndrome; and incidence of verified HRS reversal without HRS recurrence by day 30. 36.2% of patients treated with terlipressin achieved HRS reversal versus 16.8%, (P<0.001) treated with placebo. 31.7% of patients treated with terlipressin achieved HRS reversal without RRT by day 30 versus 15.8% (P=0.003) treated with placebo. The reduction in RRT requirement with terlipressin appears to extend into the post-liver transplant period, with only 9 of 46 patients (19.6%) requiring RRT post-transplant, significantly less than what was observed in the placebo group (13 of 29 patients or 44.8%), (P=0.04). A slightly lower percentage of patients in the terlipressin group received a liver transplant (23.1% [46 of 199]) compared with placebo (28.7% [29 of 101]). 24.1% of patients treated with terlipressin achieved verified HRS reversal without recurrence by day 30 versus 15.8%, (P=0.09) treated with placebo.

132/300 (44%) of subjects met systemic inflammatory response syndrome (SIRS) criteria, as defined in Example 1. Patients with overt sepsis, septic shock, or uncontrolled infection were excluded. In the SIRS subgroup, 84 patients were treated with terlipressin per the protocol in Example 1 and 48 patients were give albumin only (placebo).

Some baseline values of SIRS patients treated with terlipressin or placebo are shown in Table 1 below.

TABLE 1

| | SIRS Subgroup | SIRS Subgroup |
|---|---|---|
| | Terlipressin n = 84 | Placebo n = 48 |
| BL SCr | 3.5 (0.98) | 3.7 (1.06) |
| mg/dL(mean(SD) | (2.3-6.2) | (2.2-6.1) |
| (range) | | |
| MELD mean (SD) | 33.8 (6.27) | 33.5 (6.74) |
| CPT score mean (SD) | 10.2 (1.82) | 10.3 (2.26) |

As seen in Table 2, 33.3% of patients with SIRS and treated with terlipressin experienced HRS reversal, as compared to only 6.3% of the SIRS patients given placebo. In addition, 26.2% of patients with SIRS and treated with terlipressin experienced verified HRS reversal, as compared to only 4.2% of the SIRS patients given placebo.

TABLE 2

| | SIRS Subgroup | | |
|---|---|---|---|
| | (N = 132) | | |
| | Terlipressin | Placebo | |
| | (N = 84) | (N = 48) | |
| | n (%) | n (%) | P value |
| HRS Reversal (n, %) | 28 (33.3) | 3 (6.3) | <.001 |
| 95% CI | (0.2, 0.4) | (0.0, 0.1) | — |
| Verified HRS Reversal | 22 (26.2) | 2 (4.2) | <.001 |

Table 3 shows transplant-free survival up to 90 days for subjects with HRS reversal and/or greater than 30% improvement in serum creatinine (SCr) compared to subjects with no HRS reversal and no more than 30% improvement in SCr in the SIRS subgroup of the intent-to-treat population. 45.5% of the SIRS subgroup treated with terlipressin having HRS reversal and/or at least 30% improvement in SCr were alive and transplant-free at day 90, as compared to 28.6% for placebo. 72.7% of the SIRS subgroup treated with terlipressin having HRS reversal and/or at least 30% improvement in SCr were alive at day 90, as compared to 57.1% for placebo.

TABLE 3

| SIRS Subgroup: | | | |
|---|---|---|---|
| HRS Reversal and/or Greater | | | |
| than 30% | | | |
| Improvement in SCr | | | |
| Terlipressin | | Placebo | |
| N | Parameter | N | Parameter |

Transplant-free Survival up to
90 Days

| Survival Estimate | 33 | 0.680 | 7 | 0.536 |
|---|---|---|---|---|
| Alive and Transplant-free at | 33 | 15 (45.5) | 7 | 2 (28.6) |
| Day 90 (n, %) | | | | |
| Overall Survival up to 90 Days | | | | |
| Survival Estimate | 33 | 0.727 | 7 | 0.571 |
| Alive at Day 90 (n, %) | 33 | 24 (72.7) | 7 | 4 (57.1) |

Applying strict criteria defining HRS-1, the study demonstrated a significant reversal of worsening renal function in cirrhotic patients treated with terlipressin plus albumin when compared to those treated with albumin alone, including patients with SIRS criteria. This response was durable and associated with less need for early RRT. Therefore, terlipressin is effective in improving renal function and achieving HRS reversal in patients with HRS-1 and progressive advanced liver disease.

Although the disclosure herein has been described with reference to particular embodiments, it is to be understood that these embodiments are merely illustrative of the principles and applications of the present disclosure. It will be apparent to those skilled in the art that various modifications and variations can be made to the devices, systems, and methods of the present disclosure without departing from the spirit and scope of the disclosure. Thus, it is intended that the present disclosure include modifications and variations that are within the scope of the appended claims and their equivalents.

Reference throughout this specification to “one embodiment,” “certain embodiments,” “one or more embodiments” or “an embodiment” means that a particular feature, structure, material, or characteristic described in connection with the embodiment is included in at least one embodiment of the disclosure. Thus, the appearances of the phrases such as “in one or more embodiments,” “in certain embodiments,” “in one embodiment” or “in an embodiment” in various places throughout this specification are not necessarily referring to the same embodiment of the disclosure. Furthermore, the particular features, structures, materials, or characteristics may be combined in any suitable manner in one or more embodiments.

What is claimed is:

1. A method of reversing type 1 hepatorenal syndrome (HRS-1), the method comprising:
administering, to a patient having HRS-1, about 0.5 mg to about 1 mg of terlipressin every 6 hours for up to 3 days;
measuring serum creatinine in the patient after 3 days administration; and
comparing the measured serum creatinine to a baseline serum creatinine level, wherein:
if serum creatinine decreased by at least 30%, continue administering about 0.5 mg to about 1 mg terlipressin every 6 hours;
if serum creatinine has not decreased by 30%, administering about 1 mg to about 2 mg of terlipressin every 6 hours; and
if serum creatinine is at or above the baseline serum creatinine level, discontinue administering terlipressin.

2. The method of claim 1, wherein the terlipressin administered is terlipressin acetate.

3. The method of claim 1 further comprising continuing administration of terlipressin until 24 hours after two consecutive measured serum creatinine levels of ≤1.5 mg/dl at least 2 hours apart.

4. The method of claim 3, wherein administration of terlipressin to the patient reverses HRS-1.

5. The method of claim 1, wherein the terlipressin is administered for a maximum of 14 days.

6. The method of claim 1, wherein the terlipressin is administered as an IV bolus injection.

7. The method of claim 1, wherein administering terlipressin to the patient provides reversal of one or more complicating factors.

8. The method of claim 7, wherein reversal of one or more complicating factors reduces mortality from an associated complication within a 90 day window starting with administering the terlipressin.

9. The method of claim 1, wherein the patient does not have renal replacement therapy (RRT) post-liver transplant for at least 10 days after starting administering the terlipressin.

10. The method of claim 9, wherein the patient is alive without RRT at day 30 after starting administering the terlipressin.

11. The method of claim 1, wherein the patient has Systemic Inflammatory Response Syndrome (SIRS).

12. The method of claim 1, further comprising administering to the patient up to a maximum of 100 g per day of albumin each day.

13. A method of treating type 1 hepatorenal syndrome (HRS-1), the method comprising:
identifying a patient as having HRS-1;
administering, to a patient having HRS-1, about 0.5 mg to about 1 mg of terlipressin every 6 hours for up to 3 days;
measuring serum creatinine in the patient after 3 days administration; and
comparing the measured serum creatinine to a baseline serum creatinine level, wherein:
if serum creatinine decreased by at least 30%, continue administering about 0.5 mg to about 1 mg terlipressin every 6 hours;
if serum creatinine has not decreased by 30%, administering about 1 mg to about 2 mg of terlipressin every 6 hours; and
if serum creatinine is at or above the baseline serum creatinine level, discontinue administering terlipressin.

14. The method of claim 13, wherein the terlipressin administered is terlipressin acetate.

15. The method of claim 13, further comprising continuing administration of terlipressin until 24 hours after two consecutive measured serum creatinine levels of ≤1.5 mg/dl at least 2 hours apart.

16. The method of claim 15, wherein administration of terlipressin to the patient reverses HRS-1.

17. The method of claim 13, wherein the terlipressin is administered for a maximum of 14 days.

18. The method of claim 13, wherein the patient is administered terlipressin as an IV bolus injection.

19. The method of claim 13, wherein the patient experiences HRS reversal, verified HRS reversal, and/or greater than 30% improvement in serum creatinine.

20. The method of claim 13, wherein the patient does not have RRT post-liver transplant for at least 10 days after starting administering the terlipressin.

21. The method of claim 20, wherein the patient is alive without RRT at day 30 after starting administering the terlipressin.

22. The method of claim 13, wherein the patient has SIRS.

23. The method of claim 13, further comprising administering to the patient up to a maximum of 100 g per day of albumin each day.

24. A method of treating an adult patient with type 1 hepatorenal syndrome (HRS-1), the method comprising:
assessing a baseline serum creatinine level prior to administration of terlipressin to the patient;
initiating dosing of about 0.5 mg to about 1 mg of terlipressin to the patient every 6 hours by IV for 1-3 days;
assessing a serum creatinine level in the patient at day 4±1 day from initiating dosing; and
administering a modified dosage of terlipressin based on a comparison of the assessed serum creatinine level at day 4±1 day and the baseline serum creatinine level.

25. The method of claim 24, wherein the modified dosage is about 0.5 mg to about 1 mg terlipressin every 6 hours if serum creatinine decreased by at least 30%.

26. The method of claim 24, wherein the modified dosage is about 1 mg to about 2 mg terlipressin every 6 hours if serum creatinine has not decreased by 30%.

27. The method of claim 24, wherein the modified dosage is a discontinuation of administering terlipressin if serum creatinine is at or above the baseline serum creatinine level.

28. The method of claim 24, further comprising continuing administration until 24 hours after two consecutive serum creatinine levels of ≤1.5 mg/dL at least 2 hours apart for a maximum of 14 days.

29. The method of claim 24, wherein the terlipressin administered is terlipressin acetate.

30. The method of claim 24, wherein administration of terlipressin to the patient reverses HRS-1.

31. The method of claim 24, wherein the patient is administered terlipressin as an IV bolus injection.

32. The method of claim 24, wherein the patient experiences HRS reversal, verified HRS reversal, and/or greater than 30% improvement in serum creatinine.

33. The method of claim 24, wherein the patient does not have RRT post-liver transplant for at least 10 days after starting administering the terlipressin.

34. The method of claim 33, wherein the patient is alive without RRT at day 30 after starting administering the terlipressin.

35. The method of claim 24, wherein the patient has SIRS.

36. The method of claim 24, further comprising administering to the patient up to a maximum of 100 g per day of albumin each day.

37. A method of treating an adult patient with type 1 hepatorenal syndrome (HRS-1), the method comprising:
assessing a baseline serum creatinine level prior to administration of terlipressin to the patient;
initiating dosing of about 0.5 mg to about 1 mg of terlipressin to the patient every 6 hours by IV for 1-3 days;
assessing a serum creatinine level in the patient at day 4±1 day from initiating dosing;
administering a modified dosage of terlipressin based on a comparison of the assessed serum creatinine level at day 4±1 day and the baseline serum creatinine level; and
continuing administration until 24 hours after two consecutive serum creatinine levels of ≤1.5 mg/dL at least 2 hours apart for a maximum of 14 days.


SEQUENCE LISTING (1 sequence)

SEQ ID NO: 1 - protein, 12 aa, Artificial Sequence
  SYNTHESIZED
GKPCNQFYCGGG